(19) United States
(12) Patent Application Publication
(10) Pub. No.: US 2025/0049402 A1
(43) Pub. Date: Feb. 13, 2025

(54) METHOD FOR ASCERTAINING MATERIAL INFORMATION, X-RAY DEVICE, AND COMPUTER PROGRAM

(71) Applicant: Siemens Healthineers AG, Forchheim (DE)

(72) Inventors: Philipp Roser, Erlangen (DE); Alois Regensburger, Poxdorf (DE)

(21) Appl. No.: 18/787,090

(22) Filed: Jul. 29, 2024

(30) Foreign Application Priority Data
Aug. 11, 2023 (DE) 10 2023 207 766.3

(51) Int. Cl. A61B 6/06; A61B 6/00; A61B 6/42

(52) U.S. Cl. CPC A61B 6/06; A61B 6/4291; A61B 6/54

(57) ABSTRACT

A method is provided for ascertaining material information pertaining to a material that is present in a region of interest of an examination subject, including an imaging x-ray device having an x-ray tube assembly, an x-ray detector, and a collimator for collimating an x-ray radiation field of the x-ray tube assembly. The method includes adjusting the collimator to acquire the region of interest only, acquiring a scattered radiation image in the collimator shadow on the x-ray detector, and evaluating the scattered radiation image in order to ascertain the material information.

The present patent document claims the benefit of German Patent Application No. 10 2023 207 766.3, filed Aug. 11, 2023, which is hereby incorporated by reference in its entirety.

### TECHNICAL FIELD

The disclosure relates to a method for ascertaining material information pertaining to a material that is present in a region of interest of an examination subject, including an imaging x-ray device having an x-ray tube assembly, an x-ray detector, and a collimator for collimating an x-ray radiation field of the x-ray tube assembly. The disclosure further relates to an x-ray device and a computer program.

### BACKGROUND

In the context of medical examinations, but in particular also in the context of medical interventions, it may prove desirable in the course of x-ray imaging to be able to distinguish between different materials within an examination subject, (e.g., a patient), which materials in conventional x-ray imaging appear at least similar, in particular identical. Whereas a classical application case is the detection of calcifications, in particular also during a medical intervention that is being monitored by imaging, such a distinction may also acquire relevance with regard to blood and contrast agent. During neurological interventions on patients, the contrast agent may break through the blood-brain barrier following the intervention without any clinically significant bleeding occurring. Only the surrounding brain tissue is contaminated by contrast agent. Following such a medical intervention, it is known to produce a three-dimensional control image, for example, a C-arm CT (also called cone-beam CT). In a monoenergetic computed tomography image (CT image), but also in two-dimensional projection images, the intensity of contrast agent contaminations and actual hemorrhages cannot be differentiated.

For such cases in which it is desired to distinguish between different materials, including during medical interventions, it is generally known to employ multi-energy x-ray imaging, in particular dual-energy x-ray imaging using different x-ray energies, and/or imaging using photon-counting x-ray detectors. In any case, it involves an extreme overhead to integrate multi-energy x-ray imaging into the clinical workflow involving medical examinations and/or interventions. This is true both in terms of costs and in terms of the workflow and complexity.

### SUMMARY AND DESCRIPTION

The object underlying the disclosure is therefore to provide a method, system, or device for distinguishing different materials by x-ray imaging that may be implemented with little overhead and/or may be suitable for use in the interventional environment.

According to the disclosure, a method, an x-ray device, and a computer program are provided in order to achieve this object. The scope of the present disclosure is defined solely by the appended claims and is not affected to any degree by the statements within this summary. The present embodiments may obviate one or more of the drawbacks or limitations in the related art.

According to the disclosure, a method of the type cited in the introduction, in particular a computer-implemented method, includes: adjusting the collimator for the purpose of acquiring the region of interest only; acquiring a scattered radiation image in the collimator shadow on the x-ray detector; and evaluating the scattered radiation image in order to ascertain the material information.

A collimator shadow, in this context, is to be understood as that area on the x-ray detector in which the radiation coming from the x-ray beam source is attenuated or suppressed by the collimator on the way to the x-ray detector.

The method may be implemented by a computer, (e.g., by a control device of the x-ray device). In this case, the collimator and the acquisition arrangement composed of x-ray tube assembly and x-ray detector are actuated accordingly by the control device and an evaluation unit of the control device serves for evaluating the scattered radiation image in order to ascertain the material information. The x-ray device may use a cone-beam geometry. For example, the X-ray device may have a C-arm on which the x-ray tube assembly and the x-ray detector are arranged opposite each other. C-arm x-ray devices of this type may be used in the context of medical interventions, in particular on patients as the examination subject. The method may be employed in the context of image monitoring of a medical, in particular minimally invasive, intervention, e.g., in the context of an intervention check following the intervention. For example, a distinction between hemorrhages and pure contrast agent leakage in the brain may be made by the method after or during a neurological intervention.

The material information therefore may describe the material present in the region of interest (ROI). For example, the material information may include at least one characteristic variable and/or permit a distinction to be made between at least two possible materials in the region of interest.

One concept of the present disclosure is to acquire an x-ray image of the scattered radiation generated by the material in the region of interest. For this purpose, in a first act, a collimator of the x-ray device is aligned in such a way that the x-ray radiation field covers only the region of interest. If the x-ray detector is now also read out in the region of the collimator shadow, only scattered radiation may produce a signal there, such that, in a second act, a scattered radiation image is therefore acquired by the x-ray device. The material information pertaining to the material in the region of interest is derived based on the scattered radiation pattern in the collimator shadow. In other words, the material information may be derived based on the information pertaining to the scatter characteristics of the material in the region of interest provided by the two-dimensional scattered radiation image and in particular it is possible to distinguish between different materials or material classes. Overall, therefore, use is made of the knowledge that different materials, in particular materials of different density and/or effective atomic number, exhibit different scatter characteristics. By collimation used in a targeted manner, conditions are established in which the scatter characteristics may be measured in the form of a scattered radiation image in order to ascertain the material information by evaluation of the scattered radiation image.

In this way it is advantageously possible to dispense with performing in particular three-dimensional multi-energy imaging, in particular multi-energy CT. A classification of materials is made possible based on their scatter characteristics by acquisition of a two-dimensional scattered radiation image. A measurement involving less overhead is possible in this case.

In order to ascertain position information in respect of the region of interest, the location of the region of interest may be determined from a user input by a user and/or by a segmentation operation in at least one two-dimensional or three-dimensional prior image data set acquired by the x-ray device with positioned examination subject.

In this case, the three-dimensional location of the region of interest is beneficially established. In the case of two-dimensional prior image data sets, (e.g., projection images), different acquisition geometries, (e.g., acquisition geometries perpendicular to one another), and/or a three-dimensional prior image data set registered for this purpose and/or background information, in particular also at least one examination subject image acquired at an earlier time, may be used for determining the location in three dimensions. A user input may also be conceivable specifically for this purpose. The at least one piece of background information may also include the knowledge about a region in which an intervention has been performed on the examination subject. The location parallel to the image plane of the scattered radiation image may be used in this case for the collimation, in particular together with the location perpendicular to the image plane (distance from the x-ray tube assembly).

There are therefore several possibilities conceivable for defining the region of interest by way of its location. Thus, it is possible on the one hand to realize this at least to some extent via a user input. In this case, a user may mark the region of interest by a two-dimensional or three-dimensional visualization, in particular in at least one prior image data set already acquired with the x-ray device. Such a prior image data set may be a C-arm CT or a conventional CT image data set that was acquired for conducting an intervention check following the completion of a medical intervention. If the user is now interested in the material in a region of interest, the user may mark this by way of corresponding input device or mechanism that may be assigned to a visualization device for the prior image data set. A registration is present by definition when working with prior image data sets of the x-ray device.

It is also possible to perform a segmentation operation in a two-dimensional prior image data set (e.g., projection images) and/or a three-dimensional prior image data set (e.g., CT volumes). This may be based on a user input, or may also be fully automated, in particular on an application-oriented basis, for example, when a search may be made for specific anomalies in need of clarification with regard to the material. In this case, a three-dimensional prior image data set may be an image data set acquired for the purpose of reviewing a medical intervention. In certain examples, a semantic segmentation may be performed.

Background information may also be included in the process of determining the location of the region of interest, for example, when anatomical structures have an influence on the possible or probable location of the region of interest in the case of a patient as examination subject. For example, hemorrhages may only occur adjacent to blood vessels and may not penetrate into every type of tissue, for example, not into bone.

In the irradiation direction, (e.g., at least substantially perpendicular to the image plane of the scattered radiation image), the region of interest may not extend completely through the examination subject, but rather will account for a small portion of the total attenuation length between x-ray tube assembly and x-ray detector. This applies in particular to patients of varied build as examination subjects. Because further materials lying outside of the region of interest that are irradiated likewise emit scattered radiation, beneficial embodiments make provision to measure the desired scattered radiation from the region of interest as completely as possible and to suppress other scattered radiation. For this purpose, it is possible in particular to use an anti-scatter grid that is present anyway in many x-ray devices.

In a particularly advantageous embodiment, the x-ray device on the detector side may further have an anti-scatter grid to which an adjustment device for aligning the anti-scatter grid onto the region of interest as focus is assigned. Thus, whereas in normal operation the anti-scatter grid is in principle aligned onto a focal spot of the x-ray tube assembly so that scattered radiation from other locations is reduced, it is proposed in this embodiment to align the anti-scatter grid onto the region of interest as source of the x-ray photons that are to be measured, i.e., to perform a targeted measurement of the scattered radiation from the region of interest. In this case, the center of the region of interest in particular may be chosen as the focal spot of the region of interest, at least in the direction along the central beam. The previously discussed position information, which in fact describes the location of the region of interest (e.g., three-dimensionally), may be used to provide the basis for actuating the adjustment device. In this way, disrupting influences caused by other sources of scattered radiation may be reduced.

In a first specific embodiment variant, it may be provided in this case that the self-focusable anti-scatter grid has adjustable absorption elements, in particular strips, on which the adjustment device acts in order to achieve an at least partial alignment onto the region of interest. Flexibly focusable anti-scatter grids of this type have already been proposed in the prior art and have in particular differently adjustable and therefore focusable strips which may be adjusted, (e.g., mechanically, in particular via microactuators), and/or in some other way, (e.g., by alignment in a magnetic field).

In a second embodiment, the adjustment device has an adjustment component or apparatus for changing the position of the anti-scatter grid along the direction of the central beam, the anti-scatter grid being positioned along the direction of the central beam in order to be aligned at least partially onto the region of interest. Different embodiments are conceivable in this case, depending on whether the anti-scatter grid is at least partially movable independently or whether the anti-scatter grid may be moved together with the x-ray detector, on which it may be mounted. In this case, the adjustment device may therefore include a linear movement actuator as adjustment component, for example, a lift mechanism. During operation, the latter may also relate to the establishment of different SIDs (Source Image Distances), in particular when moving in conjunction with the x-ray detector.

In the case of a permanently focused anti-scatter grid, which during operation is aligned in a base position onto the focal spot of the x-ray tube assembly, the anti-scatter grid may be shifted from the base position by the distance between the region of interest and the focal spot of the x-ray tube assembly. In this way, the focus of the anti-scatter grid then lies in the region of interest.

In certain embodiments, it is also possible, (e.g., by repositioning the examination subject and/or the acquisition arrangement composed of x-ray tube assembly and x-ray detector), to choose the distance between the region of interest and the focal spot of the x-ray tube assembly in such a way that the freedom of movement of the anti-scatter grid is sufficient for movement around the same. In this case, it is also necessary to adjust the collimation accordingly.

In certain examples, the first and the second embodiments described above may be combined, for example, in order to compensate for an only partially realized adjustability of the anti-scatter grid itself in addition by a displacement along the direction of the central beam and the like.

In principle, if no adjustment device is present, the scattered radiation image may be acquired without an anti-scatter grid, (e.g., the anti-scatter grid is removed from the beam path for the acquisition of the scattered radiation image). This is because the anti-scatter grid would, if it cannot be aligned onto the region of interest, actually impair or even totally prevent the measurement of the scattered radiation from the region of interest, with the result that it is beneficially removed prior to the acquisition of the scattered radiation image.

The material information may advantageously include an effective atomic number and/or a density of the material and/or a classification as one of at least two candidate materials. The effective atomic number (also referred to as the effective nuclear charge (Zeff)) and/or the density of the material represent the parameters co-determining the scattering of the material. These characteristic variables may be used as material information in multi-energy imaging. In particular, when it is a question of differentiating materials, the material information may beneficially also include a classification into corresponding material classes. For example, in one application case, a distinction may be made between blood and contrast agent or between blood and calcification.

In a beneficial embodiment, it may be provided that the evaluation of the scattered radiation image includes applying at least one trained function, in particular to the scattered radiation image as input data and/or with the material information as output data.

In certain examples, a trained function maps cognitive functions that human beings associate with other human brains. By training based on training data (machine learning), the trained function has the ability to adapt to new circumstances and to detect and extrapolate patterns.

Parameters of a trained function may be adapted by training. In particular, supervised learning, semi-supervised learning, unsupervised learning, reinforcement learning, and/or active learning may be used. In addition, representation learning (also known as “feature learning”) may also be used. The parameters of the trained function may be adjusted iteratively by multiple training acts.

A trained function may include a neural network, a support vector machine (SVM), a decision tree, and/or a Bayesian network, and/or the trained function may be based on k-means clustering, Q-learning, genetic algorithms, and/or association rules. In particular, a neural network may be a deep neural network, a convolutional neural network (CNN), or a deep CNN. In addition, the neural network may be an adversarial network, a deep adversarial network, and/or a generative adversarial network (GAN). For the present application case, CNNs have proved themselves particularly suitable.

A linear evaluation and interpretation of the scattered radiation pattern revealed by the scattered radiation image may therefore be achieved by the trained function, i.e., by machine learning, in which case the scattered radiation image may be obtained as input data and the material information as output data. For example, for an application case in which two materials or material classes are to be differentiated, a binary classifier may be used.

In this case, a training process may be conducted on a large number of training data sets containing different scattered radiation patterns and associated material information. In other words, training images annotated with a ground truth may be used for training the trained function. In the present case, the training database may specifically be extended further by determining further training data sets by simulation. For example, in an advantageous development, simulations of the imaging process for the scattered radiation image may be performed for different regions of interest and/or materials in order to determine training data for specified examination subjects. An actually acquired image may therefore be used as a starting point and multiple training data sets may be generated by virtual placement of materials in at least one ROI and simulation.

Within the scope of the present disclosure, however, a similarly good evaluation may also be achieved by simulation of the imaging process of the scattered radiation image under corresponding assumptions and comparisons.

In order to ascertain the material information, similarity measures of the scattered radiation image may be determined for comparison images of different comparison material information and the material information may be ascertained based on at least one of the greatest similarity measures. In certain examples, it is conceivable in this case to have recourse to empirically determined and/or otherwise representative comparison images. Particularly advantageously, however, when a three-dimensional prior image data set of the examination subject is available, the comparison images may be determined in a targeted manner for this examination subject.

In certain examples, the comparison images may be determined at least in part by simulation of the imaging process of the scattered radiation image for a model of the examination subject, a material of the respective comparison material information being inserted in the model in the region of interest. In this case, the model may be generated from the three-dimensional prior image data set of the examination subject. The prior image data set may be a computed tomography image, in particular a C-arm CT image. In this, a material of the comparison material information is provided instead of the actually present material in order to generate the model virtually in the region of interest and a simulation is performed in order to obtain the comparison image. In order to simulate the imaging process of the scattered radiation image for the purpose of obtaining the comparison image, in which a model of the x-ray device may also be included, the acquisition parameters and/or the acquisition geometry, in particular also the collimation, of the scattered radiation image may be used accordingly. The simulation may be based on a statistical approach, (e.g., as a Monte Carlo simulation), and/or on a deterministic approach, (e.g., by solving the Boltzmann transport equation). By determining comparison images specific to the examination subject and specific to the scattered radiation image, an excellent comparison basis is created, and consequently a robust foundation is established for ascertaining the material information.

In certain examples, a plurality of scattered radiation images may be acquired using different acquisition geometries and may be evaluated in order to ascertain the material information, e.g., by statistical and/or plausibility-checking consolidation of the individual pieces of material information for each scattered radiation image. If an x-ray device is used that straightforwardly permits different acquisition geometries, scattered radiation images may accordingly be acquired from different acquisition geometries in order to obtain a broader basis for ascertaining the material information. In this connection, it is particularly beneficial if the region of interest is placed in the isocenter of the x-ray device, because then the different acquisition geometries may be set by simple rotation of the acquisition arrangement, for example, on the C-arm.

In addition to the method, the disclosure also relates to an x-ray device having an x-ray tube assembly, an x-ray detector, a collimator for collimating an x-ray radiation field of the x-ray tube assembly, and a control device. In order to ascertain material information pertaining to a material present in a region of interest of an examination subject, the control device includes: an adjustment unit for adjusting the collimator for the purpose of acquiring the region of interest only; an acquisition unit for acquiring a scattered radiation image in the collimator shadow on the x-ray detector; and an evaluation unit for evaluating the scattered radiation images in order to ascertain the material information.

The control device may have at least one processor and at least one storage device or mechanism. Functional units may be formed by software and/or hardware in order to perform the acts of the method. In other words, the control device may be configured for performing the method.

In this case, the adjustment unit and the acquisition unit may also be used respectively for adjusting the collimator and for acquiring images in other specific applications. In addition to the cited functional units, further functional units may also be provided for realizing further conceivable acts. For example, the control device may include a determination unit for determining the location of the region of interest and/or a control unit for actuating the adjustment device (or for removing the anti-scatter grid). In certain embodiments, functional units may also have subunits so that, for example, a simulation subunit of the evaluation unit may be provided.

As already described, the imaging x-ray device may be a C-arm x-ray device including a C-arm on which the x-ray tube assembly and the x-ray detector are arranged opposite each other. It is also conceivable that the x-ray device is a computed tomography (CT) device.

A computer program may be loaded directly into a storage device or component of a control device of an x-ray device and has program code that, when the computer program is executed, causes the control device to perform a method. The computer program may be stored on an electronically readable data medium, which therefore contains control information stored thereon including at least one computer program, and when the data medium is used in a control device of an x-ray device causes the control device to perform a method. The data medium may be a non-transitory data medium.

### BRIEF DESCRIPTION OF THE DRAWINGS

Further advantages and details of the present disclosure are apparent from the embodiments described in the following, as well as with reference to the drawings, in which:

FIG. 1 schematically shows an example of an acquisition geometry for acquisition of a two-dimensional x-ray image of an examination subject.

FIG. 2 schematically shows an example of an x-ray image resulting from using the acquisition geometry of FIG. 1.

FIG. 3 shows a flowchart of an embodiment of the method.

FIG. 4 schematically shows an example of an acquisition geometry with collimation onto a region of interest,

FIG. 5 shows a schematic illustrating the generation of scattered radiation in the examination subject.

FIG. 6 shows an example of a schematic illustrating the use of a focusable anti-scatter grid.

FIG. 7 shows an example of a schematic illustrating the use of an adjustable anti-scatter grid.

FIG. 8 shows an example of an x-ray device.

FIG. 9 shows the functional layout of an example of a control device of an x-ray device.

### DETAILED DESCRIPTION

Specific embodiments are explained below, with reference being made purely by way of example to the environment of a medical, (e.g., neurological), interventional procedure. It is aimed to distinguish different materials in a region of interest. One task that presents itself when reviewing the success of the intervention in the case of neurological interventions, for example, is to distinguish contrast agent that has broken through the blood-brain barrier (without bleeding) from an actual hemorrhaging (i.e., blood) as candidate materials.

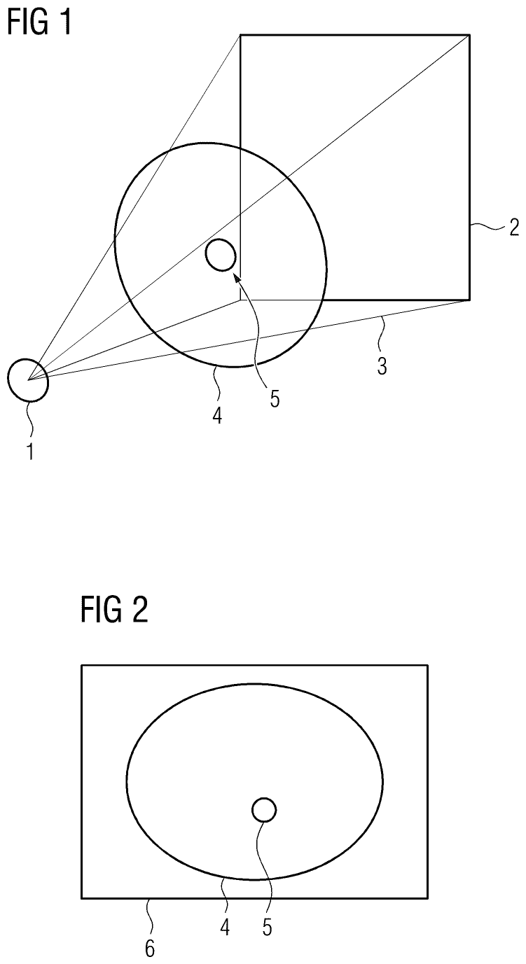

In this connection, FIG. 1 schematically shows an acquisition geometry for the acquisition of a projection image by an imaging x-ray device having an acquisition arrangement composed of an x-ray tube assembly 1 and an x-ray detector 2. Starting from a focal spot, the x-ray tube assembly 1 emits an x-ray radiation field 3 for x-raying an examination region of an examination subject 4, for example, a patient. In the example of a neurological intervention, the examination region may be the head of the patient, for example. Within the examination region in the examination subject 4, there is indicated a region of interest 5 in which a material is present which cannot be identified conclusively due to its imaging in a corresponding projection image or also in a three-dimensional image data set reconstructed from a plurality of projection images. In the above-cited example, blood and tissue-contaminating contrast agent are represented identically.

FIG. 2 schematically shows such a two-dimensional projection image 6, which within the scope of the present disclosure may be regarded as a two-dimensional prior image data set on account of its having been acquired before the method was performed. The projection image 6 shows the region of interest 5 within the examination subject 4.

In order now to be able to establish which material is present in the region of interest 5, it is proposed to analyze the scattered radiation from this region of interest 5, i.e., the material.

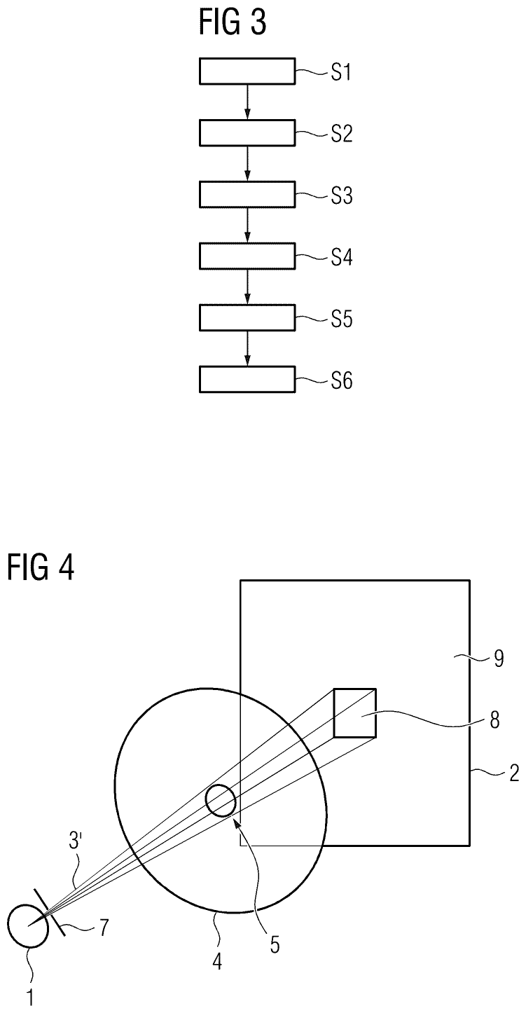

To that end, in the method explained in FIG. 3, in act S1, position information describing the location of the region of interest 5 three-dimensionally in the coordinate system of the x-ray device is initially determined. Specifically, the position information may describe dimensions at least in certain spatial directions and/or a focal point position and/or a shape, possibly also approximated, of the region of interest 5. For example, the latter may be regarded as spherical or ellipsoidal, starting from dimensions in three spatial directions that are perpendicular to one another.

On the one hand, a user input may be resorted to in order to determine the position information. For example, a user may mark the region of interest 5 in a visualization of the projection image 6, which may likewise happen in a projection image from another acquisition geometry in order to determine the three-dimensional location. A marking in a three-dimensional prior image data set is also possible.

In addition or alternatively, it is possible to segment the region of interest 5 in a two-dimensional and/or a three-dimensional prior image data set in a segmentation process, in which case a seed point predefined by a user, for example, may serve as starting point.

As a result of the position information determined in act S1, it is now possible in act S2 to actuate a collimator of the x-ray device in such a way that the x-ray radiation field 3 effective for the imaging parallel to the image plane is reduced to the region of interest 5. In other words, the field is collimated to the region of interest 5.

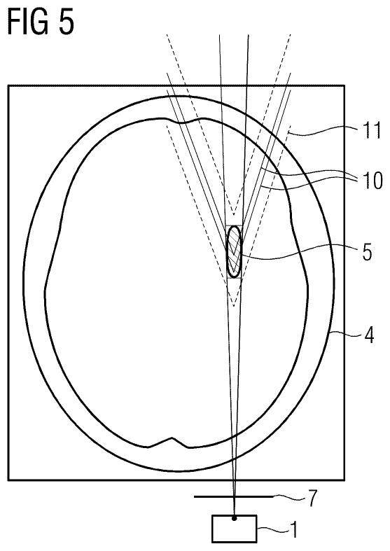

This is represented schematically in FIG. 4, which now also shows the collimator 7 arranged on the x-ray tube assembly 1. As may be seen, the effective collimated x-ray radiation field 3′ remaining behind the collimator 7 is so limited that it encloses the region of interest 5 as tightly as possible. If an image is acquired using this effective x-ray radiation field 3′, the region of interest 5 would be imaged in a central region 8 of the x-ray detector 2, while scattered radiation is measured in the surrounding collimator shadow 9.

To make this possible, an anti-scatter grid fixedly and immovably aligned onto the focal spot of the x-ray tube assembly 1 may be removed in act S3 (cf. FIG. 3 again), though in that case the scattered radiation from materials along the entire attenuation length in the examination subject 4 would be measured.

This problem is explained in more detail by the schematic diagram of FIG. 5. There, the region of interest 5 in the irradiation direction, (e.g., along the central beam), makes up only a small part of the attenuation length, with the result that unwanted scattered radiation 11 from further irradiated materials is also added to the searched-for scattered radiation 10.

In order to avoid this unwanted scattered radiation 11 as far as possible, an anti-scatter grid may be used whose focus may be directed onto the region of interest 5. In certain examples, the anti-scatter grid may be assigned an adjustment device that is actuated in act S3 in order to direct the focus of the anti-scatter grid onto the region of interest 5 and thus measure the scattered radiation 10 in a targeted manner and suppress the scattered radiation 11.

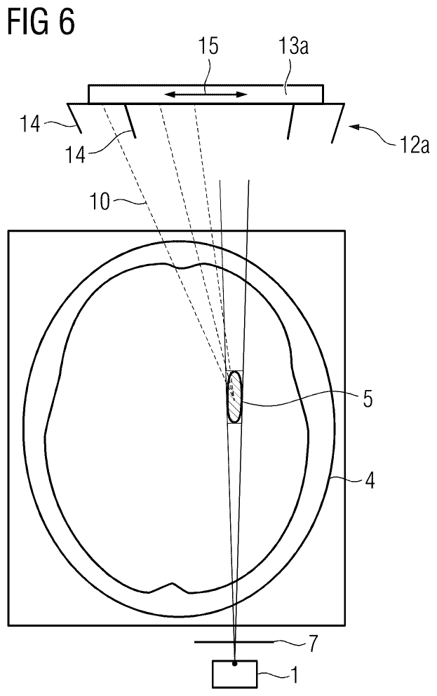

A first embodiment is explained by FIG. 6. In this case, a self-focusable anti-scatter grid 12a is used, to which an adjustment device 13a is assigned which may adjust the alignment of strips 14 (only some of which are indicated here) of the anti-scatter grid 12a such that a new focus is chosen, in the present example the position of the region of interest 5. This adjustability of the strips 14 is indicated by the arrow 15. The strips 14 are absorption elements.

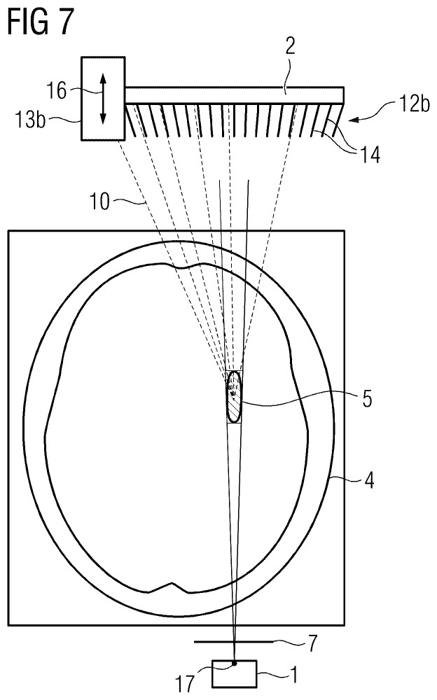

A second embodiment is explained in more detail by FIG. 7. There, the alignments of the strips 14 of the anti-scatter grid 12b are fixed. In this case, however, a modular unit composed of the x-ray detector 2 and the anti-scatter grid 12b (or alternatively the anti-scatter grid 12b alone) may be shifted along the central beam by a corresponding adjustment apparatus or component of the adjustment device 13b, as indicated by the arrow 16. Thus, if the anti-scatter grid 12b is aligned onto the focal spot 17 of the x-ray tube assembly 1 in normal operation, an alignment onto the region of interest 5 as focus may be effected in that by actuation of the adjustment device 13b the anti-scatter grid 12b is moved away from this by the distance between the focal spot 17 of the x-ray tube assembly 1 and the region of interest 5.

In principle, combinations of the first or second embodiment are also conceivable when, for example, an inadequate adjustability of the strips 14 is supplemented by a translatory movement along the central beam.

Returning to FIG. 3, in act S4, once the settings with regard to the collimator 7 and the anti-scatter grid 12a, 12b have been completed by actuation of the corresponding components, a two-dimensional scattered radiation image showing the scattered radiation from the region of interest 5 in the collimator shadow 9 may be acquired by the x-ray tube assembly 1 and the x-ray detector 2.

In act S5, the scattered radiation image is then evaluated in order to ascertain material information describing the material in the region of interest 5. The material information may include a characteristic variable of the material, e.g., an effective atomic number and/or a density. In addition or alternatively, the material information may also include a classification of the material in respect of at least two material classes, which is beneficial when a distinction needs to be made between materials.

In a first embodiment of act S5, a trained function may be used for evaluating the scattered radiation image, which trained function uses the scattered radiation image as input data and outputs the material information as output data, for example, a classification with regard to the candidate materials. The trained function has been trained using training data sets in which scattered radiation images are assigned the corresponding material information as a ground truth. At least some of the training data sets may be determined by simulation in that different materials are assumed in different regions of interest.

In a second specific embodiment variant, a three-dimensional prior image data set of the examination subject 4, which was acquired previously by the x-ray device, is taken as a starting point in order to determine comparison images for different materials by simulation. In this case, starting from the three-dimensional prior image data set, the material is inserted virtually in the region of interest in accordance with material information associated with the comparison image in order to obtain a model, and the simulation is performed. This may happen for at least two material classes/candidate materials that are to be differentiated. A similarity measure with the scattered radiation image is then determined for each of the comparison images. The material information associated with the comparison image having the highest similarity score is then determined as the evaluation result.

In act S6, the material information may be output for a user, for example, on a visualization device of the x-ray device. Storing the material information for subsequent use is also possible.

It should further be noted that it is also conceivable in exemplary embodiments to acquire a plurality of two-dimensional scattered radiation images using different acquisition geometries and to evaluate them collectively in order to ascertain the material information. In this case, acts S2 to S4 would be repeated accordingly for each acquisition geometry.

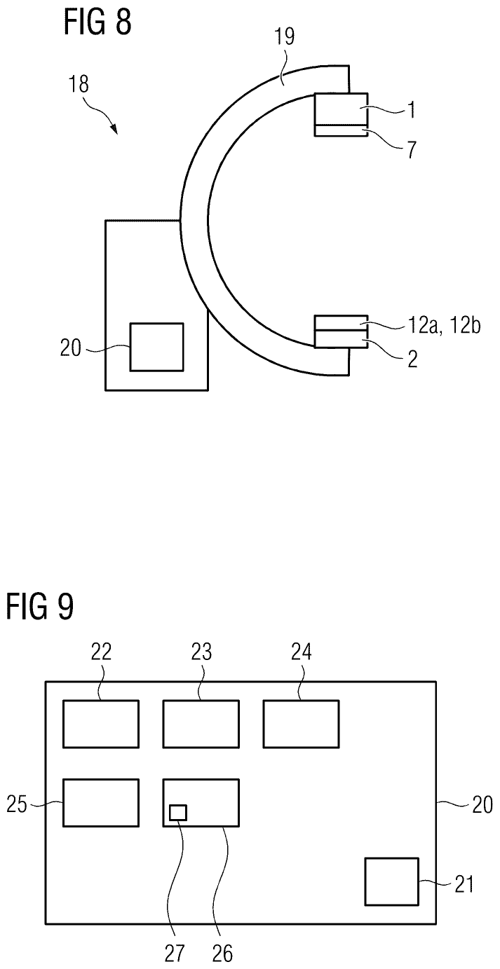

FIG. 8 shows a schematic diagram of an x-ray device 18. The x-ray device 18 includes a C-arm 19 on which the x-ray tube assembly 1 together with the collimator 7 and the x-ray detector 2 together with the anti-scatter grid 12a, 12b are arranged opposite each other. The operation of the x-ray device 18 is controlled by a control device 20, which is also embodied for performing the method.

FIG. 9 shows the functional layout of the control device 20 in greater detail. The latter firstly has a storage device 21 in which, for example, prior image data sets, scattered radiation images, material information, and the like may be stored.

For controlling the acquisition operation and for controlling the acquisition of the scattered radiation image (act S4) in particular, the control device 20 has an acquisition unit 22. Correspondingly, an adjustment unit 23 is provided for actuating the collimator 7, in particular in act S2, and a control unit 24 is provided for actuating the adjustment device 13a, 13b, in particular in act S3. A determination unit 25 permits the position information for the region of interest 5 to be determined according to act S1. Finally, an evaluation unit 26 is also provided in order to ascertain the material information according to act S5.

Optionally, when implementing the second embodiment variant, the evaluation unit 26 may also have a simulation subunit 27 for performing the corresponding simulation.

As well as the C-arm x-ray device illustrated here, the x-ray device 18 may also be an, in particular interventional, computed tomography device.

Although the disclosure has been illustrated and described in more detail on the basis of the embodiments, the disclosure is not limited by the disclosed examples and other variations may be derived herefrom by the person skilled in the art without leaving the scope of protection of the disclosure.

1. A method for ascertaining material information pertaining to a material present in a region of interest of an examination subject, comprising an imaging x-ray device having an x-ray tube assembly, an x-ray detector, and a collimator for collimating an x-ray radiation field of the x-ray tube assembly, the method comprising:
adjusting, by a control device of the imaging x-ray device, the collimator to acquire the region of interest of the examination subject only;
acquiring, by the control device, a scattered radiation image in a collimator shadow on the x-ray detector; and
evaluating, by the control device of the imaging x-ray device, the scattered radiation image in order to ascertain the material information.

2. The method of claim 1, wherein a location of the region of interest is determined from a user input by a user and/or by a segmentation process in at least one two-dimensional image data set or three-dimensional prior image data set acquired by the imaging x-ray device for the examination subject.

3. The method of claim 1, wherein the imaging x-ray device further comprises an anti-scatter grid on a detector side to which an adjustment device for aligning the anti-scatter grid onto the region of interest as focus is assigned.

4. The method of claim 3, wherein the anti-scatter grid has adjustable absorption elements on which the adjustment device acts in order to achieve an at least partial alignment onto the region of interest.

5. The method of claim 4, wherein the adjustable absorption elements comprise strips.

6. The method of claim 3, further comprising:
changing a position of the anti-scatter grid along a direction of a central beam by an adjustment component of the adjustment device,
wherein the anti-scatter grid is positioned along the direction of the central beam in order to be aligned at least partially onto the region of interest.

7. The method of claim 6, wherein the anti-scatter grid is a permanently focused anti-scatter grid, which is initially aligned in a base position onto a focal spot of the x-ray tube assembly, and
wherein the anti-scatter grid is shifted from the base position by a distance between the region of interest and the focal spot of the x-ray tube assembly.

8. The method of claim 1, wherein the scattered radiation image is acquired without an anti-scatter grid.

9. The method of claim 8, wherein the anti-scatter grid is removed from a beam path for the acquisition of the scattered radiation image.

10. The method of claim 1, wherein the material information comprises an effective atomic number, a density of the material, a classification as one of at least two candidate materials, or a combination thereof.

11. The method of claim 1, wherein the evaluating of the scattered radiation image comprises applying at least one trained function to the scattered radiation image as input data and/or with the material information as output data.

12. The method of claim 11, wherein:
training images annotated with a ground truth are used for training the trained function,
simulations of an imaging process for the scattered radiation image are performed for different regions of interest and/or materials in order to determine training data for specified examination subjects, or
a combination thereof.

13. The method of claim 1, wherein, in order to ascertain the material information, similarity measures of the scattered radiation image to comparison images of different comparison material information are determined and the material information is ascertained based on at least one of the similarity measures.

14. The method of claim 13, wherein the comparison images are determined at least in part by simulation of an imaging process of the scattered radiation image for a model of the examination subject, and
wherein a material of the respective comparison material information is inserted in the model in the region of interest.

15. The method of claim 1, wherein a plurality of scattered radiation images is acquired using different acquisition geometries and evaluated in order to ascertain the material information.

16. The method of claim 15, wherein the evaluation is by statistical and/or plausibility-checking consolidation of individual pieces of the material information for each scattered radiation image.

17. An x-ray device comprising:
an x-ray tube assembly;
an x-ray detector;
a collimator configured to collimate an x-ray radiation field of the x-ray tube assembly; and
a control device configured to:
adjust the collimator to acquire a region of interest of an examination object only;
acquire a scattered radiation image in a collimator shadow on the x-ray detector; and
evaluate the scattered radiation image in order to ascertain material information pertaining to a material present in the region of interest of the examination subject.

18. A computer program that, when executed on a control device of an imaging x-ray device, causes the control device to:
adjust a collimator of the imaging x-ray device to acquire a region of interest of an examination object only;
acquire a scattered radiation image in a collimator shadow on an x-ray detector of the imaging x-ray device; and
evaluate the scattered radiation image in order to ascertain material information pertaining to a material present in the region of interest of the examination subject.
